(19) United States
(12) Patent Application Publication
(10) Pub. No.: US 2016/0271046 A1
(43) Pub. Date: Sep. 22, 2016

(54) Antiperspirant Compositions Comprising Cyclodextrin Complexing Material

(71) Applicant: The Procter & Gamble Company, Cincinnati, OH (US)

(72) Inventors: Timothy Alan SCAVONE, Loveland, OH (US); Michael Jude LEBLANC, Cincinnati, OH (US); Lowell Alan SANKER, Montgomery, OH (US)

(21) Appl. No.: 15/166,407

(22) Filed: May 27, 2016

Related U.S. Application Data
(63) Continuation of application No. 11/712,739, filed on Mar. 1, 2007, now abandoned.

(51) Int. Cl. A61K 8/73; G06Q 30/06; A61Q 15/00; A61K 8/58; A61K 8/34

(52) U.S. Cl. CPC A61K 8/738; A61K 8/58; A61K 8/347; A61K 8/732; A61K 8/345; A61Q 15/00; G06Q 30/0629; A61K 2800/56

(57) ABSTRACT

Antiperspirant compositions are provided. An exemplary composition includes a carrier material; an antiperspirant active in an amount from about 5% to less than 19%, by weight of the composition and on an anhydrous basis; and plurality of particles comprising a cyclodextrin complexing material and a fragrance material, wherein the percent of the fragrance material that is complexed with the cyclodextrin is greater than about 75%, so that the perceptibility of the fragrance is minimized prior to its release, wherein the composition has a Residue Grade of less than about 35.

### FIELD OF THE INVENTION

The present invention is directed to topical antiperspirant compositions.

### SUMMARY OF THE INVENTION

The present invention provides antiperspirant compositions employing cyclodextrin-fragrance complexes that are formed by a spray-drying process and/or have particular characteristics, such as relatively low moisture levels or relatively low levels of unbound fragrance. Complex particles having relatively low moisture levels are believed to have less of a tendency to agglomerate prior to their inclusion into a composition, which can lead to a smoother feeling composition instead of a grainy or gritty feeling composition. This is particularly important for rub-on type antiperspirant compositions.

At least some of the antiperspirant compositions of the present invention are designed such that the fragrance complexed with the cyclodextrin is substantially imperceptible (“hidden”) to a consumer prior to the happening of a triggering event, such as perspiration. This feature mitigates against the fragrance bleeding through prematurely to alter the initial scent expression provided by a separate neat fragrance or to simply provide an unwanted initial scent expression in products marketed as, for example, unscented, scent-free, hypoallergenic, sensitive, or the like. These antiperspirant compositions can be enabled by cyclodextrin-fragrance complexes having relatively small amounts of unbound fragrance.

In accordance with one of the preferred embodiments, there has now been provided an antiperspirant composition comprising a carrier material; an antiperspirant active in an amount from about 5% to less than 19%, by weight of the composition and on an anhydrous basis; and a plurality of particles comprising a cyclodextrin complexing material and a fragrance material, wherein the percent of the fragrance material that is complexed with the cyclodextrin is greater than about 75%, so that the perceptibility of the fragrance is minimized prior to its release.

In accordance with another preferred embodiment, there has now been provided an antiperspirant composition comprising a carrier material; an antiperspirant active in an amount from about 5% to less than 19%, by weight of the composition on an anhydrous basis; and a plurality of particles comprising a cyclodextrin complexing material and a fragrance material, the plurality of particles having a moisture level, before inclusion into the composition, of less than about 20% by weight of the particles.

In accordance with yet another preferred embodiment, there has now been provided an antiperspirant composition comprising a carrier material; an antiperspirant active in an amount from about 5% to less than 19%, by weight of the composition and on an anhydrous basis; and a plurality of particles comprising a cyclodextrin complexing material and a fragrance material, wherein the plurality of particles are formed using a process comprising a step of spray drying.

The above compositions may be formulated to yield a low residue level; for example, to exhibit a Residue Grade (as defined herein) of less than about 35. The compositions may comprise an emollient system for minimizing residue. Exemplary systems comprise one or more emollients having a refractive index of greater than or equal to 1.4460.

In accordance with another preferred embodiment, there has now been provided an antiperspirant composition comprising a carrier material; a fragrance material; and a scent-releasing system comprising a combination of cyclodextrin complexing material and corn starch.

The antiperspirant compositions of the present invention may be manufactured into various product forms, including, but not limited to, solids, soft solids, roll-ons, and aerosols.

The present invention is also directed to methods of merchandising antiperspirant compositions. One exemplary embodiment comprises the step of communicating a comparison to perspective buyers between a first antiperspirant composition comprising cylcodextrin-fragrance complexes and a second antiperspirant composition comprising corn starch. The comparison may comprise, for example, a performance-related comparison, a scent or fragrance comparison, and/or a protection-related comparison.

### DETAILED DESCRIPTION OF THE INVENTION

The present invention may be understood more readily by reference to the following detailed description of illustrative and preferred embodiments. It is to be understood that the scope of the claims is not limited to the specific features, methods, conditions, or parameters described herein, and that the terminology used herein is for the purpose of describing particular embodiments by way of example only and it not intended to be limiting of the claimed invention.

Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

The term “ambient conditions,” as used herein, refers to surrounding conditions at about one atmosphere of pressure, 50% relative humidity and about 25° C.

As used herein, “comprising” means that other steps and other ingredients which do not affect the end result can be added. This term encompasses the terms “consisting of” and “consisting essentially of”. The compositions of the present invention can comprise, consist of, and consist essentially of the elements and limitations of the invention described herein, as well as any of the additional or optional ingredients, components, steps, or limitations described herein.

The term “anhydrous” as used herein means that the deodorant composition of the present invention, and the essential or optional components thereof, are substantially free of added or free water. For example, the deodorant compositions of the present invention may comprise less than about 2%, less than about 1%, less than about 0.5%, or zero percent of free or added water, by weight of the composition.

As used herein, the term “antiperspirant composition,” includes antiperspirant compositions, deodorant compositions, body sprays, and the like. The product form is unlimited unless specified otherwise.

Antiperspirant compositions of the present invention include a liquid carrier, an antiperspirant active (and/or deodorant), and a plurality of particles comprising a cyclodextrin complexing material and a fragrance material. These features will be discussed below, followed by a description of optional ingredients that may or may not be employed in the antiperspirant compositions of the present invention.

### I. LIQUID CARRIER

Compositions of the present invention include a liquid carrier. Suitable liquid carriers include, but are not limited to, any topically safe and effective organic, silicone-containing or fluorine-containing, volatile or non-volatile, polar or non-polar liquid carrier. The liquid carrier is preferably liquid under ambient conditions, and can include one or more liquid carrier materials provided that the any such combination of materials is in liquid form under ambient conditions. Depending on the type of product form desired, concentrations of the liquid carrier in the compositions will typically range from about 10% or from about 30% to about 90% or to about 75%, by weight of the composition.

Nonlimiting examples of suitable liquid carriers include C₁ to C₂₀ monohydric alcohols, i.e., C₂ to C₈ monohydric alcohols; C₂ to C₄₀ dihydric or polyhydric alcohols, i.e., C₂ to C₂₀ dihydric or polyhydric alcohols; alkyl ethers of all such alcohols, i.e. C₁-C₄ alkyl ethers; and polyalkoxylated glycols, i.e. propylene glycols and polyethylene glycols having from 2 to 30 repeating alkoxylate (e.g., ethoxylate or propoxylate) groups and polyglycerols having from 2 to 16 repeating glycerol moieties; their derivatives and mixtures thereof.

Specific examples of such alcohol liquid carriers include propylene glycol, hexylene glycol, dipropylene glycol, tripropylene glycol, glycerin, propylene glycol methyl ether, dipropylene glycol methyl ether, ethanol, n-propanol, n-butanol, t-butanol, 2- methoxyethanol, 2-ethoxyethanol, ethylene glycol, isopropanol, isobutanol, 1,4-butylene glycol, 2,3-butylene glycol, trimethylene glycol, 1,3- butanediol, 1,4,-butanediol, propylene glycol monoisostearate, PPG-3 myristyl ether, PEG-4 (also known as PEG-200), PEG-8 (also known as PEG-400), 1,2, pentanediol, PPG-14 butylether, dimethyl isosorbide, and combinations thereof. Other similar but suitable solvents for use as liquid carriers are described, for example, in U.S. Pat. No. 4,781,917, issued to Luebbe et al., Nov. 1, 1998, U.S. Pat. No. 5,643,558, issued to Provancal et al., Jul. 1, 1997, U.S. Pat. No. 4,816,261, issued to Luebbe et al., Mar. 28, 1989 and EP 404 533 A1, published Dec. 27, 1990 by Smith et al..

The antiperspirant compositions of the present invention may comprise a silicone liquid carrier. The concentration of the silicone liquid carrier may range from about 10% or from about 15% of a silicone liquid carrier, by weight of the composition to about 90% or to about 65% of a silicone liquid carrier, by weight of the composition. The silicone liquid carriers suitable for use herein may include volatile or non-volatile silicones.

Nonlimiting examples of suitable silicone liquid carriers for use herein include those volatile silicones that are described in Todd et al., “Volatile Silicone Fluids for Cosmetics”, Cosmetics and Toiletries, 91:27-32 (1976). Suitable amongst these volatile silicones include the cyclic silicones having from about 3 or from about 4 to about 7 or to about 6, silicon atoms. Specifically are those which conform to the formula:

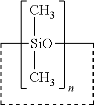

wherein n is from about 3, from about 4 or about 5 to about 7 or to about 6. These volatile cyclic silicones generally have a viscosity value of less than about 10 centistokes. Other suitable silicone liquid carriers for use herein include those volatile and nonvolatile linear silicones which conform to the formula:

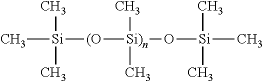

wherein n is greater than or equal to 0. The volatile linear silicone materials will generally have viscosity values of less than 5 centistokes at 25° C. The non-volatile linear silicone materials will generally have viscosity values of greater than 5 centistokes at 25° C.

Specific examples of suitable volatile silicones for use herein include, but are not limited to, hexamethyldisiloxane; Silicone Fluids SF-1202 and SF-1173 (commercially available from G.E. Silicones); Dow Corning 244, Dow Corning 245, Dow Corning 246, Dow Corning 344, and Dow Coming 345, (commercially available from Dow Corning Corp.); Silicone Fluids SWS-03314, SWS-03400, F-222, F-223, F-250, and F-251 (commercially available from SWS Silicones Corp.); Volatile Silicones 7158, 7207, 7349 (available from Union Carbide); Masil SF-V™ (available from Mazer); and mixtures thereof.

Specific examples of suitable non-volatile linear silicones for use herein include, but are not limited to, Rhodorsil Oils 70047 available from Rhone-Poulenc; Masil SF Fluid available from Mazer; Dow Coming 200 and Dow Coming 225 (available from Dow Corning Corp.); Silicone Fluid SF-96 (available from G.E. Silicones); Velvasil™ and Viscasil™ (available from General Electric Co.); Silicone L-45, Silicone L-530, and Silicone L-531 (available from Union Carbide); and Siloxane F- 221 and Silicone Fluid SWS-101 (available from SWS Silicones).

Other suitable non-volatile silicone liquid carriers for use in the antiperspirant compositions of the present invention include, but are not limited to, non-volatile silicone emollients such as polyalkylarylsiloxanes, polyestersiloxanes, polyethersiloxane copolymers, polyfluorosiloxanes, polyaminosiloxanes, and combinations thereof. These non-volatile silicone liquid carriers will generally have viscosity values of less than about 100,000 centistokes, less than about 500 centistokes, or from about 1 centistokes to about 200 centistokes or to about 50 centistokes, as measured under ambient conditions.

Other suitable liquid carriers for use in the compositions of the present invention include, but are not limited to, organic liquid carriers such as mineral oil, petrolatum, isohexadecane, isododecane, various other hydrocarbon oils, and mixtures thereof. Preferred are mineral oil and branched chain hydrocarbons having from about 4 or from about 6 carbon atoms to about 30 or to about 20 carbon atoms. Specific non-limiting examples of suitable branched chain hydrocarbon oils include isoparaffins available from Exxon Chemical Company as Isopar C™ (C7-C8 Isoparaffin), Isopar E™ (C8-C9 Isoparaffin), Isopar G™ (C10-11 Isoparaffin), Isopar H™ (C11-C12 Isoparaffin), Isopar L™ (C11-C13 Isoparaffin), Isopar M™ (C13-C14 Isoparaffin), and combinations thereof. Other nonlimiting examples of suitable branched chain hydrocarbons include Permethyl™ 99A (isododecane), Permethyl™ 102A (isoeicosane), Permethyl™ 101A (isohexadecane), and combinations thereof. The Permethyl™ series are available from Preperse, Inc., South Plainfield, N.J., U.S.A. Other non-limiting examples of suitable branched chain hydrocarbons include petroleum distillates such as those available from Phillips Chemical as Soltrol™ 130, Soltrol™ 170, and those available from Shell as Shell Sol™ 70, -71, and -2033, and mixtures thereof.

Examples of other suitable organic liquid carriers include the Norpar™ series of paraffins available from Exxon Chemical Company as Norpar™ 12, -13, and -15; octyldodecanol; butyl stearate; diisopropyl adipate; dodecane; octane; decane; C₁-C₁₅ alkanes/cycloalkanes available from Exxon as Exxsol™ D80; C₁₂-C₁₅ alkyl benzoates available as Finsolv-TN™ from Finetex; and mixtures thereof. Other suitable liquid carriers include benzoate co-solvents, cinnamate esters, secondary alcohols, benzyl acetate, phenyl alkane, and combinations thereof.

The antiperspirant compositions of the present invention may be formulated as an aqueous or anhydrous composition. Aqueous compositions may comprise from about 10% or from about 15% water, by weight of the composition to about 75%, to about 60%, or to about 50% water, by weight of the composition. Anhydrous compositions may comprise less than about 10%, less than about 3%, less than about 1%, or zero percent water, by weight of the composition.

### II. ANTIPERSPIRANT ACTIVE

Compositions of the present invention contain an antiperspirant active. Exemplary concentrations of the antiperspirant active include from about 0.5% to about 60%, and more preferably from about 5% to about 35%, by weight of the composition. In one preferred embodiment of the present invention, the antiperspirant active is included at a level of from about 5% to less than 19%, by weight of the composition. In another preferred embodiment, the antiperspirant active is employed at a level of from about 10% to less than or equal to about 15%, by weight of the composition. These weight percentages are calculated on an anhydrous metal salt basis exclusive of water and any complexing agents such as, for example, glycine and glycine salts. The antiperspirant active as formulated in the composition are typically in the form of dispersed particulate solids having a preferred average particle size or equivalent diameter of less than about 100 microns, more preferably less than about 20 microns, and even more preferably less than about 10 microns.

The antiperspirant active for use in the compositions of the present invention may include any compound, composition or other material having antiperspirant activity. By way of example only, the antiperspirant actives may include astringent metallic salts, especially inorganic and organic salts of aluminum, zirconium and zinc, as well as mixtures thereof. Particular antiperspirant active examples include, but are not limited to, aluminum-containing and/or zirconium-containing salts or materials, such as aluminum halides, aluminum chlorohydrate, aluminum hydroxyhalides, zirconyl oxyhalides, zirconyl hydroxyhalides, and mixtures thereof.

Aluminum salts useful in the present invention include those that conform to the formula:

Al₂(OH)ₐCl_{b}.xH₂O

wherein a is from about 0 to about 5; the sum of a and b is about 6; x is from about 1 to about 8; where a, b, and x may have non-integer values. For example, aluminum chlorohydroxides referred to as “¾ basic chlorohydroxide,” wherein a is about 4.5; “⅚ basic chlorohydroxide”, wherein a=5; and “⅔ basic chlorohydroxide”, wherein a=4 may be used. Processes for preparing aluminum salts are disclosed in U.S. Pat. No. 3,887,692, issued to Gilman on Jun. 3, 1975; U.S. Pat. No. 3.904,741, issued to Jones et al. on Sep. 9, 1975; and U.S. Pat. No. 4,359,456 issued to Gosling et al. on Nov. 16, 1982. A general description of these aluminum salts can also be found in Antiperspirants and Deodorants, Cosmetic Science and Technology Series Vol. 20, 2nd edition, edited by Karl Laden. Mixtures of aluminum salts are described in British Patent Specification 1,347,950, filed in the name of Shin et al. and published Feb. 24, 1974.

Zirconium salts for use in the present invention include those which conform to the formula:

ZrO(OH)₂₋ₐClₐ.xH₂O

wherein a is from about 0.5 to about 2; x is from about 1 to about 7; where a and x may both have non-integer values. These zirconium salts are described in Belgian Patent 825,146, issued to Schmitz on Aug. 4, 1975. Useful to the present invention are zirconium salt complexes that additionally contain aluminum and glycine, commonly known as “ZAG complexes”. These complexes contain aluminum chlorohydroxide and zirconyl hydroxy chloride conforming to the above-described formulas. Such ZAG complexes are described in U.S. Pat. No. 4,331,609, issued to Orr on May 25, 1982 and U.S. Pat. No. 4,120,948, issued to Shelton on Oct. 17, 1978.

Compositions of the present invention may alternatively or additionally comprise a deodorant active. Suitable deodorant actives may be selected from the group consisting of antimicrobial agents (e.g., bacteriocides, fungicides), malodor-absorbing material, and combinations thereof. For example, antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof.

### III. CYCLODEXTRIN COMPLEXING MATERIAL

Compositions of the present invention include a cyclodextrin complexing material for substantially “hiding” a fragrance material until a triggering mechanism has occurred, such as, for example, perspiration, to “release” the fragrance material. As used herein, the term “cyclodextrin” includes any of the known cyclodextrins such as unsubstituted cyclodextrins containing from about six to about twelve glucose units, especially alpha-cyclodextrin, beta-cyclodextrin, gamma-cyclodextrin and/or their derivatives and/or mixtures thereof. For example, the present invention may use cyclodextrins selected from the group consisting of beta-cyclodextrin, hydroxypropyl alpha-cyclodextrin, hydroxypropyl beta-cyclodextrin, methylated-alpha-cyclodextrin, methylated-beta-cyclodextrin, and mixtures thereof. Cyclodextrins may be included within the compositions from at least about 0.1%, from at least about 1%, from at least about 2%, or from at least about 3% to about 25%, to about 20%, to about 15% or to about 10%, by weight of the composition.

Cyclodextrin particles and cyclodextrin complexes comprising a fragrance material can be formed by various methods. For example, a solvent (e.g., water), unloaded cyclodextrin particles, and a fragrance material can be placed into a container and then mixed for a period of time to permit loading of fragrance molecules into “cavities” of cyclodextrin molecules. The mixture may or may not be processed further; e.g., processed through a colloid mill and/or homogenizer. The solvent is then substantially removed from the resulting mixture or slurry to yield cyclodextrin-fragrance complex particles. Different manufacturing techniques may however impart different particle/complex characterizations, which may or may not be desirable in a final composition. In accordance with some of the preferred embodiments of the present invention, the particles and/or complexes have a low level of moisture prior to their inclusion into the composition. For a given volume of cyclodextrin particles (at least some of which being complexed with a fragrance material), it is preferred to have a moisture level of less than about 20% by weight of the particles, more preferred to have a moisture level of less than about 10% by weight of the particles, and even more preferred to have a moisture level of less than about 6% by weight of the particles, prior to the inclusion of the volume of particles into the composition. Other moisture levels may be suitable for compositions of the present invention; accordingly, these preferred levels should not be read into claims that do not specify a cyclodextrin particle/complex moisture level.

Spray drying a slurry or mixture of cyclodextrin-fragance complexes is one manufacturing technique capable of producing the cyclodextrin particles and cyclodextrin complexes having the above-noted, preferred moisture levels. Table I below provides a comparison of spray dried cyclodextrin complexes versus complexes formed via an extruder process (kneading).

TABLE I
Cyclodextrin Complex Moisture Level

| Sample | % Moisture |
|---|---|
| Spray Dry Process Sample A | 4.4 |
| Spray Dry Process Sample B | 3.7-4.5 |
| Spray Dry Process Sample C | 5.3 |
| Extruder Process Sample A | 27.87 |
| Extruder Process Sample B | 27.97 |
| Extruder Process Sample C | 24.00 |

Water content, USP (United States Pharmacopeia, current as of Aug. 1, 2006) <921> Method I is the analytical method for determining cyclodextrin complex moisture level, as shown in Table I.

As one can see from Table 1, the moisture level directly manifested by these two methods is dramatically different. It should be understood that this comparison is not intended to disclaim kneading/extruder processes from appended claims that do not specify a particular complex formation process. Rather, a kneading and extrusion method, or other method forming particles/complexes with higher than desired moisture levels, would require additional processing after their initial formation. For example, extruded complexes may require processing through an oven or dryer, or exposure to a controlled environment for a period of time.

Although not wishing to be bound by theory, it is believed that cyclodextrin particles/complexes having a relatively high moisture level have an increased tendency to agglomerate. The agglomerated particles may reach a size so as to become perceptible by a consumer; that is, a consumer may characterize the composition as being “gritty.” And a “gritty” antiperspirant composition may not be desirable to some consumers, particular in solid product forms where the product is rubbed against the body as the means of applying the antiperspirant. Microbial growth is another potential disadvantage associated with employing cyclodextrin particles/complexes with relatively high moisture levels into a final composition depending on the remaining ingredients of the composition and/or storage parameters.

The efficiency or level of complexing with a fragrance material is another parameter of cyclodextrin complexes that can vary greatly depending on the manufacturing techniques employed. Put another way, the percent of fragrance material that is associated with the interior of a cyclodextrin molecule compared to the percent of fragrance material that is associated with the exterior of the cyclodextrin complex. The fragrance material that is on the exterior region of the complex is essentially free to be expressed without the requirement of a triggering mechanism, such as perspiration. The probability that a consumer perceives the fragrance material prior to a triggering mechanism increases as the level of free fragrance increases. And perception of a fragrance material prior to a triggering mechanism may not be desired depending on the overall composition design and targeted benefit associated with employment of the cyclodextrin complexes. In accordance with at least some of the preferred embodiments, the percent of fragrance material that is complexed with cyclodextrin is greater than about 75%, in some instances greater than about 90%, and in other instances greater than about 95%. It should be understood that these levels of fragrance complexation are directly associated with the complex formation process itself; the percentages do not represent a formulation design of adding a first percentage of fragrance material via a cyclodextrin complex and adding a second percentage of neat fragrance material.

Spray drying a slurry or mixture of cyclodextrin-fragance complexes is one manufacturing technique capable of producing cyclodextrin complexes having the above-noted levels of fragrance complexation. Table II below provides a comparison of spray dried cyclodextrin complexes versus complexes formed via an extruder process (kneading).

TABLE II
Percent of Fragrance Loading in Cyclodextrin Complexes

| Sample | Complexation Efficiency |
|---|---|
| Spray Dry Process Sample A | 96.6 |
| Spray Dry Process Sample B | 96.8 |
| Spray Dry Process Sample C | 96.2 |
| Extruder Process Sample A | 60.77 |
| Extruder Process Sample B | 65.47 |
| Extruder Process Sample C | 67.07 |

One can see from Table II that spray drying is capable of producing cyclodextrin complexes with very little free fragrance as compared to a kneading/extruder process. The skilled artisan should appreciate that the comparison provided in Table II is not intended to disclaim kneading/extruder processes from appended claims that do not specify a particular complex formation process. Rather, additional processing steps may, for example, need to be employed to eliminate free fragrance associated with extruded complexes prior to their inclusion into a composition.

The analytical method for determining the percent of fragrance complexed, as shown in Table II, determines the free fragrance level in the complex by dissolving a sample in tetrahydrofuran (THF) adding an internal standard, and analyzing by capillary gas chromatography (GC). The complexed fragrance level is measured by extracting the same sample in acetone containing an internal standard, and analyzing by GC.

Complexation Efficiency=% Complexed/[%Complexed+% Free]

### Original Sample Preparation

### Internal Standard Stock Solution (ISSS)

Weigh 0.625 g±0.05 g of Diphenyloxide into a tared 100 mL volumetric flask and make to volume with acetone (Baker HPLC grade 9254-03). This is a suggested internal standard, other materials may be substituted as necessary to avoid chromatographic overlap depending on the specific fragrance to be analyzed.

### Standards

Select a sufficient number (typically 10-20) of fragrance components to account for 80% or greater of the total area of the fragrance chromatogram. A synthetic blend of these components will be the primary standard used to quantitate fragrance levels. A sample of the fragrance is used as the secondary standard which enables correction for the fact that less than 100% of the components are calibrated.

### Primary Standard Calibration Solutions

Primary stock: Weigh 0.1 g (to 0.001g) of the individual fragrance components to be quantitated into a tared 100 mL volumetric flask and record the weights. Make to volume with acetone. Pipette 3.0 mL of the primary stock into a 50 mL volumetric flask and add 0.50 mL of ISSS for complexed calibration standard and dilute to volume with acetone. Pipette 3.0 mL of the primary stock into a 50 mL volumetric flask and add 0.50 mL of ISSS for neat calibration standard and dilute to volume with THF (Baker 9450-03).

### Secondary Fragrance Standard Calibration Solutions

Secondary stock: Weigh 0.5 g (±0.1 g with precision to 0.0001 g) of the fragrance into a tared 100 mL volumetric flask and record weight. Make to volume with extraction solution for total fragrance (acetone); mix well. Pipette 3.0 mL of the secondary stock into a 50 mL volumetric flask and add 0.50 mL of ISSS for complexed fragrance standard and dilute to volume with acetone. Pipette 3.0 mL of the secondary stock into a 50 mL volumetric flask and add 0.50 mL of ISSS for neat fragrance standard and dilute to volume with THF.

### Preparation of Samples

The ASE Solvent Extractor used in these analyses was a Dionex 200. Insert fiber filter (Dionex #49458) into an 11 mL cell body (Dionex part number 47004) with end cap on one end. Push filter to meet end cap. Tare on balance. Carefully add 1.000 gram (+/−0.250 grams) of sample to cell and record actual weight. Using a funnel, add sand (30-40 mesh, EM Science EM-SX0075-1 or alternate inert material) to fill the cell, place another fiber filter on top and close cell with second end cap. Use care in applying this filter so it is not above the end of the cell but rather push down slightly so the filter is inside the walls of the cell. This is to avoid filter particles from accumulating within the threads of the end caps which can cause leaking during extraction. Record cell serial number to correspond with sample identification. Load the cells and their corresponding collection vials (60 ml Dionex 48784) onto the ASE. [Note: For each sample two collection vials will be needed, one for the THF extraction (neat fragrance) and one for the acetone extraction (complex fragrance). To extract multiple samples it is recommended that all the THF extractions be done prior to the acetone extractions due to the temperature difference between the two methods.]

### ASE Methods

| THF - Neat Fragrance | Extraction | Acetone - Complexed |
|---|---|---|
| 0 min | Preheat | 0 min |
| 5 min | Heat | 6 min |
| 4 min | Static | 15 min |
| 100% | Flush | 100% |
| 60 sec | Purge | 60 sec |
| 1 | Cycles | 3 |
| 500 psi | Pressure | 2000 psi |
| 40° C. | Temperature | 110° C. |
| 100% THF | Solvent | 100% Acetone |

### Preparation of ASE

Assure sufficient nitrogen flow by verifying pressures for solvent bottles are at 10 psi, system air is at 50 psi and compression oven is at 130 psi. Verify there is an adequate amount of nitrogen to complete the run. Typically 1000 psi of nitrogen is used to extract 15 samples. Enter ASE methods, above, and save each method under a separate number. For example: The THF method can be saved as number 1 and the acetone method can be saved as number 2. Verify there is an adequate volume of both THF and acetone present to complete the run. Approximately 30 mL of each solvent is used per sample (note: usage can vary from system to system). With rinse collection vials present and an adequate volume of THF present, rinse the system with THF a few times to prime lines and remove any air. With cells and their corresponding labeled collection vials in place, the ASE methods are ready to begin.

### Post ASE Sample Preparation for Complexed Fragrance (In Acetone)

Remove ASE collection vials containing complexed fragrance extract. Screw off the cap on the collection vial. Add 0.50 mL of ISSS directly to the collection vial, with a volumetric pipet. Add approximately 30 mL of acetone. Replace the cap onto the collection vial tightly. Shake well for approximately 30 seconds.

### Post ASE Sample Preparation for Neat Fragrance (in THF)

Remove ASE collection vials containing neat fragrance extract. Screw off the cap on the collection vial. Add 0.50mL of ISSS directly to the collection vial, with a volumetric pipet. Add approximately 30mL of tetrahydrofuran. Replace the cap onto the collection vial tightly. Shake well for approximately 30 seconds.

### Apparatus Criteria (Suggested Type or Source)

Gas Chromatograph HP5890 or equivalent equipped with capillary inlet system. and flame ionization detector with peak integration capabilities

Column DB-5 column, 30 m×0.32 mm I D with 1.0 micron coating, J&W Scientific cat. no. 123-5033

### Gas Chromatographic Conditions

Carrier Gas Helium UHP grade or regular grade helium purified through a dry tube and an oxygen scrubber. Flow-pressure regulated at 15 psi with 30 mL/min split flow.

Oven Temperature. 50° C.-250° C. @ 6° C./min; 250° C.-315° C. @ 70° C./min ; Hold at 315 oC for 5 minutes

Injector Temperature 250° C.

Detector Temperature 325° C.

Hydrogen and Air Flows Optimized for gas chromatograph used

Integration Threshold 2, peak width 0.04

Injection 1 microliter: splitless mode

### Calulations

% Analyte=[(AvRe (A)(B)×100]/[C×D] Where:

AvRf=Average response factor for standard sample

A=Weight of internal standard added to sample solution

B=Area of analyte peak in sample chromatogram

C=Area of internal std. peak in sample chromatogram

D=Sample weight in gram

100=Factor for percent conversion

Corrected % Complexed or % Free in samples=[sum of the % of all individual fragrance components in sample×100]/ [sum of the % of all individual fragrance components in the sec. std.]

The cyclodextrin complexes may be coated to minimize premature release/activation. Generally, any material that is capable of resisting water penetration is suitable. The coating material may include, for example, hydrocarbons, waxes, petrolatum, silicones, silicone derivatives, partially or fully esterfied sucrose esters, and polyglycerol esters. Using petrolatum as an example, a coating process may include combining cyclodextrin complexes with petrolatum at a ratio of about 1:1, for example, and then mixing until the complexes are satisfactorily coated. Another technique for delaying release or activation of a complexed fragrance, as contemplated herein, is to combine the fragrance material with an occlusive ingredient, such as, for example, coconut oil or petrolatum, before complexing with cyclodextrin. And the fragrance material and the cyclodextrin-fragrance complex may both be coated in some instances.

Coating cyclodextrin-fragrance complexes is one technique that may permit inclusion of the complexes into polar composition matrices, such as, for example, deodorant sticks, clear gels, or clear antiperspirant sticks. That is, antiperspirant/deodorant products containing water, propylene glycol, dipropylene glycol, or other solvent with a C Log P value of less than about 2.

A scent-releasing system may be employed in the antiperspirant compositions, wherien the system comprises cyclodextrin complexing material, as described above, in combination with other complexing or encapsulating materials known to the skilled artisan. For example, a scent-releasing system may be employed comprising a combination of cyclodextrin complexing material and one or more additional encapsulating materials. Exemplary encapsulating materials include starches, oligosaccharides, polyethylenes, polayamides, polystyrenes, polyisoprenes, polycarbonates, polyesters, polyacrylates, vinyl polymers, silicas, and aluminosilicates. Commercially available encapsulating materials N-Lok™, manufactured by National Starch, Narlex™ (ST and ST2), and Capsul E™ are useful for the present invention. These materials comprise pregelatinized waxy maize starch and optionally, glucose. The starch is modified by adding monofunctional substituted groups such as octenyl succinic acid anhydride. Accordingly, compositions of the present invention may include a neat fragrance material, a cyclodextrin-fragrance complex, and fragrance material encapsulated with materials other than cyclodextrin, such as those described above. The fragrances of this three-component scent-releasing system may be the same or different. Combining different scent-releasing technologies permits customization of scent expression profiles.

It should be understood that compostions of the present invention may optionally employ “unloaded” cyclodextrin particles to act as a scavenger for malodor. These optional cyclodextrin particles may or may not have similar properties (or be manufactured using the same techniques) as the complexes described above.

### IV. FRAGRANCE MATERIAL

Compositions of the present invention employ at least one fragrance material that is complexed with the cyclodextrin complexing material discussed above. A representative, non-limiting, list of fragrance materials that may be complexed with the cyclodextrin includes anethole, benzaldehyde, decyl aldehyde, benzyl acetate, benzyl alcohol, benzyl formate, benzyl propionate, iso-bornyl acetate, camphene, cis-citral (neral), citronellal, citronellol, citronellyl acetate, paracymene, decanal, dihydrolinalool, dihydromyrcenol, methyl benzyl carbinyl acetate, dimethyl benzyl carbinyl acetate, dimethyl phenyl carbinol, eucalyptol, helional, geranial, geraniol, geranyl acetate, geranyl nitrile, cis-3-hexenyl acetate, dihydrocitronellal, d-limonene, linalool, linalool oxide, tetra-hydro linalool, alpha-methyl ionone, methyl nonyl acetaldehyde, methyl phenyl carbinyl acetate, laevo-menthyl acetate, menthone, iso-menthone, myrcene, myrcenyl acetate, myrcenol, nerol, neryl acetate, nonyl acetate, phenyl ethyl alcohol, phenyl acetaldehyde, alpha-pinene, beta-pinene, gamma-terpinene, terpineol, alpha-terpineol, beta-terpineol, terpinyl acetate, vertenex (para-tertiary-butyl cyclohexyl acetate), gamma-methyl ionone, undecalactone, undecylenic aldehyde, alpha-damascone, beta-damascone, amyl acetate, lemon oil, orange oil, and mixtures thereof.

It may be desirable to include only a single fragrance material (may include a combination of perfumes or other aromatic materials) in the antiperspirant composition, and for that fragrance material to be complexed with cyclodextrin. For these embodiments, the intent is for the consumer not to perceive (or only minimally perceive) the fragrance material upon application of the antiperspirant composition. Such compositions may be marketed with the following terms: unscented, scent-free, sensitive, and/or hypoallergenic. During use, perspiration would release the fragrance material enabling it to be perceived by the consumer.

On the other hand, it may be desirable to include two or more fragrance materials in the antiperspirant composition, with at least one the fragrance materials being complexed with the cyclodextrin complexing material and at least one other fragrance material being added as a neat fragrance into the composition. In these embodiments, it is preferred for the complexed and neat fragrances to be different from one another. The differences can include types and numbers of perfumes or other aromatic materials employed in the individual fragrance materials, the concentration level, or both.

The neat or non-complexed fragrance material may include the materials delineated above, or may include other perfumes/aromatic materials known to a person of ordinary skill in the art of creating fragrances. Typical fragrances are described in Arctander, Perfume and Flavour Chemicals (Aroma Chemicals), Vol. I and II (1969) and Arctander, Perfume and Flavour Materials of Natural Origin (1960). U.S. Pat. No. 4,322,308, issued to Hooper et al., March 30, 1982 and U.S. Pat. No. 4,304,679, issued to Hooper et al., Dec. 8, 1981 disclose suitable fragrance materials including, but not limited to, volatile phenolic substances (such as iso-amyl salicylate, benzyl salicylate, and thyme oil red), essence oils (such as geranium oil, patchouli oil, and petitgrain oil), citrus oils, extracts and resins (such as benzoin siam resinoid and opoponax resinoid), “synthetic” oils (such as Bergamot™ 37 and Bergamot™ 430, Geranium™ 76 and Pomeransol™ 314); aldehydes and ketones (such as B-methyl naphthyl ketone, p-t-butyl-A-methyl hydrocinnamic aldehyde and p-t-amyl cyclohexanone), polycyclic compounds (such as coumarin and beta-naphthyl methyl ether), esters (such as diethyl phthalate, phenylethyl phenylacetate, non-anolide 1:4).

Optional neat fragrances also include esters and essential oils derived from floral materials and fruits, citrus oils, absolutes, aldehydes, resinoides, musk and other animal notes (e.g., natural isolates of civet, castoreum and musk), balsamic, and alcohols (such as dimyrcetol, phenylethyl alcohol and tetrahydromuguol). For example, the present invention may comprise fragrances selected from the group consisting of decyl aldehyde, undecyl aldehyde, undecylenic aldehyde, lauric aldehyde, amyl cinnamic aldehyde, ethyl methyl phenyl glycidate, methyl nonyl acetaldehyde, myristic aldehyde, nonalactone, nonyl aldehyde, octyl aldehyde, undecalactone, hexyl cinnamic aldehyde, benzaldehyde, vanillin, heliotropine, camphor, para-hydroxy phenolbutanone, 6-acetyl 1,1,3,4,4,6 hexamethyl tetrahydronaphthalene, alpha-methyl ionone, gamma-methyl ionone, amyl-cyclohexanone, and mixtures thereof.

Other suitable fragrances are those which mask or help to mask odors associated with perspiration (also referred to herein as odor masking fragrances), some non-limiting examples of which are described in U.S. Pat. No. 5,554,588, issued to Behan et al., Sep. 10, 1996, U.S. Pat. No. 4,278,658, issued to Hooper et al., Dec. 8, 1981, U.S. Pat. No. 5,501,805, issued to Behan et al., Mar. 26, 1996, and EP Patent Application 684 037 A1, published Nov. 29, 1995, by Gordon et al..

### V. SUSPENDING/THICKENING AGENT

The solid antiperspirant compositions of the present invention may also comprise thickening agents to help provide the composition with the desired viscosity, rheology, texture and/or product hardness, or to otherwise help suspend any dispersed solids or liquids within the composition. The term “thickening agent” may include any material known or otherwise effective in providing suspending, gelling, viscosifying, solidifying or thickening properties to the composition or which otherwise provide structure to the final product form. These thickening agents may include gelling agents, polymeric or nonpolymeric agents, inorganic thickening agents, or viscosifying agents. The thickening agents may include organic solids, silicone solids, crystalline or other gellants, inorganic particulates such as clays or silicas, or combinations thereof.

The concentration and type of the thickening agent selected for use in the antiperspirant composition of the present invention will vary depending upon the desired product form, viscosity, and hardness. The thickening agents suitable for use herein, may have a concentration range from at least about 0.1%, at least about 3%, or at least about 5% but no more than about 35%, no more than about 20%, or no more than about 10%, by weight of the composition.

Non-limiting examples of suitable gelling agents of the present invention include fatty acid gellants, salts of fatty acids, hydroxyl acids, hydroxyl acid gellants, esters and amides of fatty acid or hydroxyl fatty acid gellants, cholesterolic materials, dibenzylidene alditols, lanolinolic materials, fatty alcohols, triglycerides, sucrose esters such as SEFA behenate, inorganic materials such as clays or silicas, other amide or polyamide gellants, and mixtures thereof.

Suitable gelling agents include fatty acid gellants such as fatty acid and hydroxyl or alpha hydroxyl fatty acids, having from about 10 to about 40 carbon atoms, and ester and amides of such gelling agents. Non-limiting examples of such gelling agents include, but are not limited to, 12-hydroxystearic acid, 12-hydroxylauric acid, 16-hydroxyhexadecanoic acid, behenic acid, eurcic acid, stearic acid, caprylic acid, lauric acid, isostearic acid, and combinations thereof.

Preferred gelling agents are 12-hydroxystearic acid, esters of 12-hydroxystearic acid, amides of 12-hydroxystearic acid and combinations thereof.

Other suitable gelling agents include amide gellants such as disubstituted or branched monoamide gellants, monsubstituted or branched diamide gellants, triamide gellants, and combinations thereof, including n-acyl amino acid derivatives such as n-acyl amino acid amides, n-acyl amino acid esters prepared from glutamic acid, lysine, glutamine, aspartic acid, and combinations thereof. Other suitable amide gelling agents are described in U.S. Pat. No. 5,429,816, issued Jul. 4, 1995, and U.S. Pat. No. 5,840,287, filed Dec. 20, 1996.

Still other examples of suitable gelling agents include fatty alcohols having at least about 8 carbon atoms, at least about 12 carbon atoms but no more than about 40 carbon atoms, no more than about 30 carbon atoms, or no more than about 18 carbon atoms. For example, fatty alcohols include but are not limited to cetyl alcohol, myristyl alcohol, stearyl alcohol and combinations thereof.

Non limiting examples of suitable tryiglyceride gellants include tristearin, hydrogenated vegetable oil, trihydroxysterin (Thixcin® R, available from Rheox, Inc.), rape seed oil, castor wax, fish oils, tripalmitin, Syncrowax® HRC and Syncrowax® HGL-C (Syncrowax® available from Croda, Inc.).

Other suitable thickening agents include waxes or wax-like materials having a melt point of above 65° C., more typically from about 65° C. to about 130° C., examples of which include, but are not limited to, waxes such as beeswax, carnauba, bayberry, candelilla, montan, ozokerite, ceresin, hydrogenated castor oil (castor wax), synthetic waxes and microcrystalline waxes. Castor wax is preferred within this group. Other high melting point waxes are described in U.S. Pat. No. 4,049,792, Elsnau, issued Sep. 20, 1977.

Further thickening agents for use in the solid antiperspirant compositions of the present invention may include inorganic particulate thickening agents such as clays and colloidal pyrogenic silica pigments. For example, colloidal pyrogenic silica pigments such as Cab-O-Sil®, a submicroscopic particulated pyrogenic silica may be used. Other known or otherwise effective inorganic particulate thickening agents that are commonly used in the art can also be used in the solid antiperspirant compositions of the present invention.

Suitable clay thickening agents include montmorillonite clays, examples of which include bentonites, hectorites, and colloidal magnesium aluminum silicates. These and other suitable clays may be hydrophobically treated, and when so treated will generally be used in combination with a clay activator. Non-limiting examples of suitable clay activators include propylene carbonate, ethanol, and combinations thereof.

### VI. OPTIONAL INGREDIENTS

The antiperspirant compositions may further comprise one or more optional components which may modify the physical or chemical characteristics of the compositions or serve as additional “active” components when deposited on the skin. Of course, such optional components may be included provided that they are physically and chemically compatible and do not otherwise unduly impair product stability, aesthetics, or performance. Nonlimiting examples of such optional materials include, but are not limited to, pH buffering agents, additional malodor controlling agents such as deodorant actives, fragrance materials, emollients (e.g., glycerin), humectants, soothing agents, dyes and pigments, medicaments, baking soda and related materials, preservatives, and soothing agents such as aloe vera, allantoin, D-panthenol, pantothenic acid derivatives (e.g., those disclosed in U.S. Pat. No. 6,495,149), avocado oil and other vegetative oils, and lichen extract.

Antioxidants are another class of materials that may optionally be employed in the antiperspirant compositions of the present invention. A representative, non-limiting, list of suitable antioxidants includes amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example .alpha.-carotene, .beta.-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, .gamma -linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa-and hepta-thionine sulfoximine) in very small compatible dosages (for example pmole to .mu.mole/kg), also (metal) chelators (for example (.alpha.-hydroxyfatty acids, palmitic acid, phytic acid, lactoferrine), .alpha.-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example .gamma.-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, (.alpha.-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxy-butyrophenone, uric acid and derivatives thereof, mannose and derivatives thereof, Superoxid-Dismutase, zinc and derivatives thereof (for example ZnO, ZnSO.sub.4), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

### VII. RESIDUE GRADE

The antiperspirant compositions of the present invention may optionally be formulated to provide low residue performance These compositions may have a Residue Grade of less than about 50, than about 40, or less than about 35. In this context, the Residue Grade is an indirect measure of the visible residue that is likely to remain on the skin after topical application of the antiperspirant composition.

The Residue Grade is determined by the Naugahyde Method. In accordance with this method, a piece of commercial, black, dull finished, small grained vinyl (Boltaflex vinyl upholstery, Prefixx protective finish, Mfr. GenCorp Polymer Products) cut to a 10 cm×15 cm rectangular strip is placed on a horizontal platform. Each corner of the vinyl strip is then secured with a small binder clip after the material has been slightly stretched to create a smooth surface. A antiperspirant composition under ambient conditions (for at least 24 hours prior to testing) is trimmed flat across the top of the container and placed on a balance which is then tared to 0.00 grams in order to determine the mass of product to be applied to the vinyl. The antiperspirant composition contained within and partially extending out 0.5 cm from a conventional antiperspirant package (5.2 cm×2.7 cm topographically oval package) is positioned perpendicular to and above the positioned vinyl by securing the container onto a movable mechanical arm, such that the flat, trimmed surface of the secured product extends out of the package and is facing parallel to the horizontally positioned vinyl. The antiperspirant composition is then slowly moved vertically toward the vinyl sample until the flat, trimmed surface of the product rests upon the far left area of the positioned vinyl. A weight is placed on the product sample so that the entire flat, trimmed surface of the product uniformly contacts the positioned vinyl during testing. The applied weight is selected so as to provide 45.3 grams/cm² to the trimmed surface of the product sample, e.g., 500 gram weight applied to an oval 5.2 cm×2.7 cm trimmed surface area. The weighted sample is then manually moved repeatedly back and forth across the entire length of the piece of vinyl at a rate of one stroke per second (one stroke equals one left to right movement or one right to left movement), until 0.20 gm.+/−0.02 gm. of product has been evenly applied over 15.24 cm×5.08cm area of the black vinyl (0.0026 grams of product per cm² of the black vinyl surface). The product sample is then removed from the mechanical arm piece and weighed. The vinyl is then unclipped and carefully removed from the platform and dried down for 6 hours.

A calibrated Minolta CR-300 Chroma Meter (available from Minolta Corp., Ramsey, N.J., USA) is then used to measure the L-value (on the L, a, b color scale) of each of the applied vinyl surfaces. For each of the applied vinyl surfaces, twelve random, non-overlapping areas of the applied surface are measured for L-values by the Chroma Meter with its clear plastic view port removed to allow direct placement of the Meter port onto the vinyl so that the meter port is positioned over but without touching the applied vinyl surface. An average L-value is then determined for the twelve measurements which then corresponds to the Residue Grade as described herein.

Incorporating emollients having a relatively high refractive index (e.g., 1.4460 or higher) is one technique for reducing residue/whitening effects of antiperspirant compositions. Exemplary non-volatile, non-silicone emollient materials falling within this category include isostearyl isostearate, glycereth-7-benzoate, C12-C15 alkyl benzoate, octyldodecyl benzoate, isostearyl lactate, isostearyl palmitate, benzyl laurate, laureth 4, laureth 7, oleth 2, PEG 4, PEG 12, PPG 2 ceteareth 9, PPG 2 isodeceth 12, PPG 5 butyl ether, PPG 14 butyl ether, PPG 15 butyl ether, PPG 53 butyl ether, octyldodecanol, and polydecene. Exemplary, non-volatile silicone emollient materials include phenyltrimethicone and dimethicone copolyol. Mixtures of the above emollient materials may also be employed.

### VIII. METHOD OF MERCHANDISING ANTIPERSPIRANT COMPOSITIONS

The present invention is also directed to methods of merchandising consumer products, including, but not limited to, antiperspirant/deodorant products. The methods can be employed to market a single product or an array of products. The methods generally comprise the step of communicating a comparison between at least two different products. Such comparisons can relate to product attributes (negative and/or positive), targeted uses, product “side effects”, and efficacy. For example, comparisons between two different antiperspirant products may relate to performance, scent, fragrance, efficacy (e.g., wetness protection and/or odor control), skin feel, residue, and/or skin irritation. The information contained in the comparison is unlimited, but preferably (although not required) should include an identification of the consumer care product along with trigger words such, as “compare to” or “compare with” followed by at least a portion of the brand name (or other recognizable identifier) of the comparative product. Comparisons may be made via packaging, advertisements, brochures, product displays, and coupons, for example.

In accordance with one of the preferred embodiments, there has now been provided a method of merchandising antiperspirant products comprising the step of communicating a comparison to perspective buyers between a first antiperspirant/deodorant composition comprising cyclodextrin-fragrance complexes and a second antiperspirant/deodorant composition comprising corn starch or other encapsulating technology that is different than cyclodextrin complexing material.

### IX. EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the invention.

### Examples 1 and 2

### Invisible Solid Antiperspirant Sticks

| Ingredient | Example 1, wt % | Example 2, wt % |
|---|---|---|
| Cyclopentasiloxane | Quantity Sufficient | Quantity Sufficient |
| Al Zr Trichlorohydrex | 14.50 | 17.50 |
| Glycinate | | |
| PPG-14 Butyl Ether | 8.00 | 0 |
| Isopropyl Palmitate | 0 | 10.00 |
| Mineral Oil | 0 | 2.00 |
| Stearyl Alcohol | 12.00 | 12.00 |
| C12-C15 Alkyl Benzoate | 5.00 | 0 |
| Spray Dried Cyclodextrin | 3.00 | 2.54 |
| Fragrance Complex | | |
| Castor Wax | 2.50 | 2.50 |
| Neat Perfume | 1.25 | 1.25 |
| Fumed Silica | 0.60 | 0.60 |

Examples 1 and 2 can be made as follows: combine the waxes (Castor Wax, Stearyl alcohol) with the emollients in a suitable container and heat the mixture while stirring to 75° C. Next, add the powders (antiperspirant active, beta cyclodextrin fragrance complex, and silica) and mix. The mixture can then be homogenized with a dispersator to fully disperse the powders. Add the neat perfume and then cool the mixture to about 60° C. Lastly, pour the cooled mixture into antiperspirant canisters. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An antiperspirant composition, comprising:
(a) a carrier material;
(b) an antiperspirant active in an amount from about 5% to less than 19%, by weight of the composition on an anhydrous basis; and
(c) a plurality of particles comprising a cyclodextrin complexing material and a fragrance material, wherein the percent of the fragrance material that is complexed with the cyclodextrin is greater than about 75%, so that the perceptibility of the fragrance is minimized prior to its release,
wherein the composition has a Residue Grade of less than about 35.

2. The antiperspirant composition of claim 1, wherein the antiperspirant active is included in an amount from about 10% to less than or equal to about 15%, by weight of the composition on an anhydrous basis.

3. The antiperspirant composition of claim 1, wherein the percent of the first fragrance material that is complexed with the cyclodextrin is greater than about 90%.

4. The antiperspirant composition of claim 1, wherein the percent of the first fragrance material that is complexed with the cyclodextrin is greater than about 95%.

5. The antiperspirant composition of claim 1, further comprising a second fragrance material that is not complexed with the cyclodextrin complexing material, and that is different from the fragrance material.

6. The antiperspirant composition of claim 5, wherein the difference between the first fragrance material and the second fragrance material is selected from the group consisting of concentration level, chemical make-up, and combinations thereof.

7. The antiperspirant composition of claim 1, further comprising an antioxidant.

8. The antiperspirant composition of claim 1, wherein the antioxidant is BHT.

9. The antiperspirant composition of claim 1, further comprising corn starch.

10. The antiperspirant composition of claim 1, further comprising glycerin.

11. The antiperspirant composition of claim 1, wherien the composition is in a form selected from the group consisting of solid, soft solid, roll-on and aerosol.

12. An antiperspirant composition, comprising:
(a) a carrier material;
(b) an antiperspirant active in an amount from about 5% to less than 19%, by weight of the composition on an anhydrous basis;
(c) a plurality of particles comprising a cyclodextrin complexing material and a fragrance material, wherein the percent of the fragrance material that is complexed with the cyclodextrin is greater than about 75%, so that the perceptibility of the fragrance is minimized prior to its release; and
(d) an emollient system for minimizing residue arising from the cyclodextrin, the emollient system comprising one or more emollients having a refractive index of greater than or equal to 1.4460.

13. The antiperspirant composition of claim 12, wherein the antiperspirant active is included in an amount from about 10% to less than or equal to about 15%, by weight of the composition on an anhydrous basis.

14. The antiperspirant composition of claim 12, wherein the percent of the first fragrance material that is complexed with the cyclodextrin is greater than about 90%.

15. The antiperspirant composition of claim 12, wherein the percent of the first fragrance material that is complexed with the cyclodextrin is greater than about 95%.

16. The antiperspirant composition of claim 12, further comprising a second fragrance material that is not complexed with the cyclodextrin complexing material, and that is different from the fragrance material.

17. The antiperspirant composition of claim 16, wherein the difference between the first fragrance material and the second fragrance material is selected from the group consisting of concentration level, chemical make-up, and combinations thereof.

18. An antiperspirant composition, comprising:
(a) a carrier material;
(b) an antiperspirant active in an amount from about 5% to less than 19%, by weight of the composition on an anhydrous basis; and
(c) a plurality of particles comprising a cyclodextrin complexing material and a fragrance material, the plurality of particles having a moisture level, before inclusion into the composition, of less than about 20% by weight of the particles,
wherein the composition has a Residue Grade of less than about 35.

19. The antiperspirant composition of claim 18, wherein the antiperspirant active is included in an amount from about 10% to less than or equal to about 15%, by weight of the composition on an anhydrous basis.

20. The antiperspirant composition of claim 18, wherein the plurality of particles has a moisture level, before inclusion into the composition, of less than about 10% by weight of the plurality of particles.

21. The antiperspirant composition of claim 18, wherein the plurality of particles has a moisture level, before inclusion into the composition, of less than about 6% by weight of the plurality of particles.

22. The antiperspirant composition of claim 18, further comprising a second fragrance material that is not complexed with cyclodextrin.

23. The antiperspirant composition of claim 22, wherein the second fragrance material is different from the fragrance material.

24. The antiperspirant composition of claim 18, wherien the composition is in a form selected from the group consisting of solid, soft solid, roll-on, and aerosol.

25. An antiperspirant composition, comprising:
(a) a carrier material;
(b) an antiperspirant active in an amount from about 5% to less than 19%, by weight of the composition on an anhydrous basis;
(c) a plurality of particles comprising a cyclodextrin complexing material and a fragrance material, the plurality of particles having a moisture level, before inclusion into the composition, of less than about 20% by weight of the particles; and
(d) an emollient system for minimizing residue arising from the cyclodextrin, the emollient system comprising one or more emollients having a refractive index of greater than or equal to 1.4460.

26. An antiperspirant composition, comprising:
(a) a carrier material;
(b) an antiperspirant active in an amount from about 5% to less than 19%, by weight of the composition on an anhydrous basis; and
(c) a plurality of particles comprising a cyclodextrin complexing material and a fragrance material, wherein the plurality of particles are formed using a process comprising a step of spray drying,
wherein the composition has a Residue Grade of less than about 35.

27. The antiperspirant composition of claim 26, wherein the antiperspirant active is included in an amount from about 10% to less than or equal to about 15%, by weight of the composition on an anhydrous basis.

28. The antiperspirant composition of claim 26, further comprising a second fragrance material that is not complexed with cyclodextrin.

29. The antiperspirant composition of claim 28, wherein the second fragrance material is different from the fragrance material.

30. An antiperspirant composition, comprising:
(a) a carrier material;
(b) an antiperspirant active in an amount from about 5% to less than 19%, by weight of the composition on an anhydrous basis;
(c) a plurality of particles comprising a cyclodextrin complexing material and a fragrance material, wherein the plurality of particles are formed using a process comprising a step of spray drying; and
(d) an emollient system for minimizing residue arising from the cyclodextrin, the emollient system comprising one or more emollients having a refractive index of greater than or equal to 1.4460.

31. An antiperspirant composition, comprising:
(a) a carrier material;
(b) a fragrance material; and
(c) a scent-releasing system comprising a combination of cyclodextrin complexing material and corn starch.

32. A method of merchandising antiperspirant or deodorant compositions, comprising the step of communicating a comparison to perspective buyers between a first antiperspirant/deodorant composition comprising cyclodextrin-fragrance complexes and a second antiperspirant/deodorant composition comprising a scent-releasing or fragrance encapsulating technology that does not comprise cyclodextrin material.

33. The method of claim 32, wherien the scent-releasing or fragrance encapsulating technology that does not comprise cyclodextrin material comprises corn starch.

34. The method of claim 32, wherien the comparison comprises a performance comparison.

35. The method of claim 32, wherein the comparison comprises a scent or fragrance comparison.

36. The method of claim 32, wherein the comparison comprises a protection comparison.
